(19) United States
(12) Patent Application Publication
(10) Pub. No.: US 2007/0007462 A1
(43) Pub. Date: Jan. 11, 2007

(54) Large area detectors and displays

(76) Inventors: Robert Stevens, Wiltshire (GB); Geoffrey Howard Grayer, Berkshire (GB); Andrew Tulloch, Berkshire (GB); Norman Leslie Pratt, Hertfordshire (GB); Ejaz Huq, Oxfordshire (GB)

Correspondence Address: BIRCH STEWART KOLASCH & BIRCH, PO BOX 747, FALLS CHURCH, VA, 22040-0747, US

(21) Appl. No.: 10/551,646

(22) PCT Filed: Apr. 1, 2004
(86) PCT No.: PCT/GB04/01439

§ 371 Date: Jul. 21, 2006

(30) Foreign Application Priority Data
Apr. 1, 2003 (GB) 0307526.4
Jul. 9, 2003 (GB) 0316082.7
Jul. 24, 2003 (GB) 0317386.1

(51) Int. Cl. G01T 1/24

(52) U.S. Cl. 250/370.14

(57) ABSTRACT

A large area detector (3) for use, for example, as an x-ray detector for medical imaging consists of a radiation sensitive detector (8) in combination with a monolithic amplification device consisting of a plurality of alternately stacked dynode and insulation layers having channels (6) extending therethrough in communication with respective signal collectors (10). The large area detector (3) is capable of being formed in tiles of 1 m² or more whilst providing high resolution of the order of 50-60 pixels per mm².

The present invention relates to large area detectors and displays employed in, but not limited to, medicine.

Significant advances have been made in medical technology in relation to x-ray imaging. Traditionally, x-ray images have been taken using x-ray sensitive films as the recording medium. However, more recently solid state detectors have been proposed which use amorphous silicon in combination with a scintillator to generate digital x-ray images.

An example of a solid state detector that is capable of providing two dimensional position sensitive information is described in US2003/0080298. The detector is in the form of a silicon avalanche photodiode (APD) which, for the purposes of x-ray detection, would be used in combination with a scintillator. However, there are limits to both the resolution of such a detector and its total detecting area (300-400 mm² tile). Hence, there are still significant technical problems to be overcome before solid state detectors of this type could be used in large area detectors.

The possibility of using solid state detectors of the type describe above has been considered in the field of medical imaging and in U.S. Pat. No. 6,263,043 a combined MR and X-ray scanner is described which envisages a solid state radiation detector mounted within the patient bed that is also used for MR scanning. Although U.S. Pat. No. 6,263,043 illustrates a solid state detector having a surface area sufficient to image almost half the length of a man, as was mentioned above this is not practicable with currently available solid state sensors.

The present invention therefore seeks to provide a monolithic electromagnetic radiation detector that is particularly suited to implementation as a large area detector. The present invention also seeks to provide a large area x-ray detector for use in medical diagnosis and treatment. The present invention also separately seeks to provide a monolithic large area display.

Reference herein to electromagnetic radiation detectors is intended to encompass but not be limited to the detection of gamma rays, x-rays, bioluminescence and visible light including ultra violet wavelengths.

The present invention provides an electromagnetic radiation detector comprising a layer of a radiation sensitive material; an amplification device; and one or more signal collectors, the amplification device comprising a plurality of alternatively stacked layers of a dynode material and an electrical insulator, each dynode layer having exposed secondary electron emissive material and each stacked layer having a plurality of apertures which align with apertures in adjacent layers to form a plurality of electron multiplier channels extending through the stacked layers, and power supply connections to each dynode layer for applying a predetermined voltage potential to each dynode layer wherein the one or more signal collectors is positioned at the opposite end of the electron multiplier channels to the radiation sensitive material such that a signal from the radiation sensitive material of the detection of electromagnetic radiation is amplified in one or more of the electron amplifier channels before being collected by said one or more signal collectors.

In one aspect the present invention provides an x-ray imaging device comprising a layer of x-ray radiation sensitive material; an amplification device; and an image processor, the amplification device comprising a plurality of alternatively stacked layers of a dynode material and an electrical insulator, each dynode layer having exposed secondary electron emissive material and each stacked layer having a plurality of apertures which align with apertures in adjacent layers to form a plurality of electron multiplier channels extending through the stacked layers, power supply connections to each dynode layer for applying a predetermined voltage potential to each dynode layer and a plurality of anodes located at the ends of the electron multiplier channels, each anode being associated with one or more channels and having an image data link for supplying position sensitive image data to the image processor for generating a two dimensional image of x-ray radiation incident on said imaging device.

The x-ray imaging device is preferably capable of an image resolution of at least 10 pixels per mm, more preferably 50 pixels per mm and ideally is mounted within a patient bed.

In an alternative embodiment the present invention provides a display comprising a layer of phosphor material; an amplification device; a plurality of field emission tips; and a driver device, the amplification device comprising a plurality of alternatively stacked layers of a dynode material and an electrical insulator, each dynode layer having exposed secondary electron emissive material and each stacked layer having a plurality of apertures which align with apertures in adjacent layers to form a plurality of electron multiplier channels extending through the stacked layers, power supply connections to each dynode layer for applying a predetermined voltage potential to each dynode layer wherein electrons emitted by the field emission tips under control of the driver device are multiplied by the amplification device before being incident on the layer of phosphor material so as to generate a two dimensional image in the layer of phosphor material.

Ideally the display has a minimum tile size of 1 m².

Embodiments of the present invention will now be described by way of example only with reference to the accompanying drawings, in which:

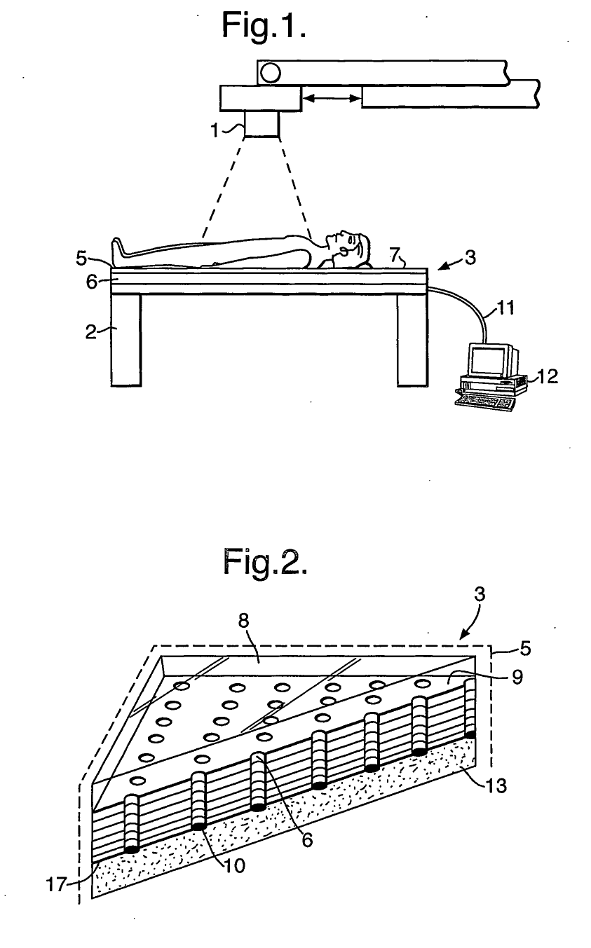

FIG. 1 illustrates schematically an x-ray imaging device for use in medical diagnosis in accordance with the present invention;

FIG. 2 is an enlarged cut-away perspective view of a portion of the x-ray detector of FIG. 1;

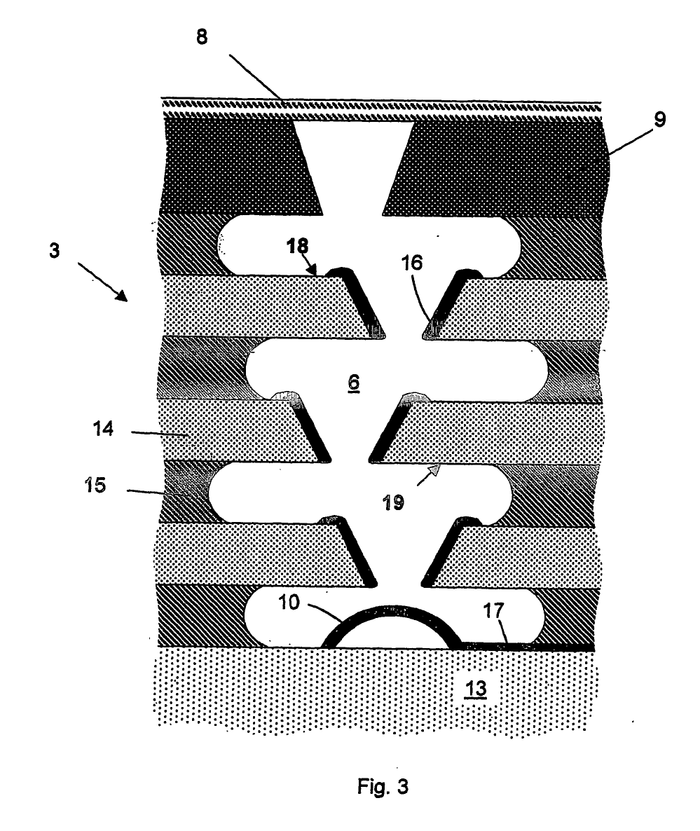

FIG. 3 is a schematic cross sectional view of a single channel of the x-ray detector of FIG. 1;

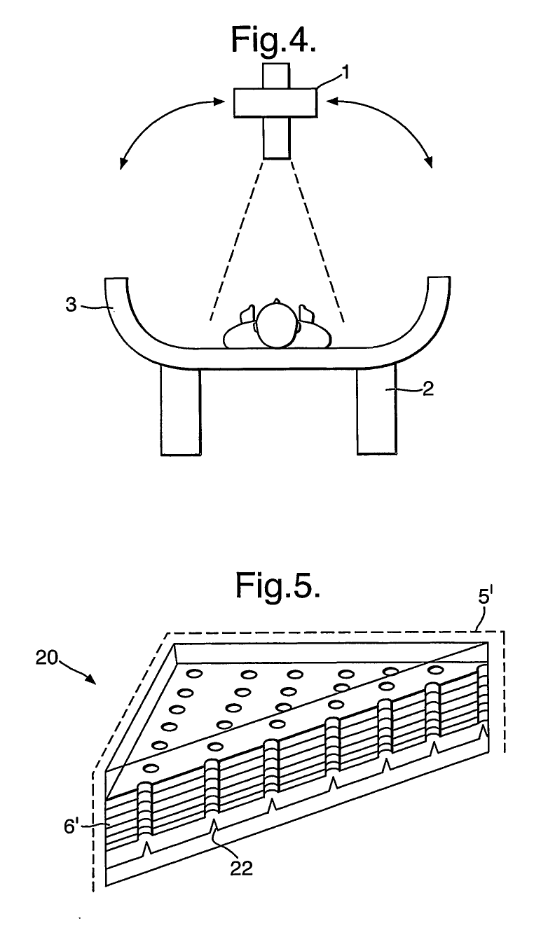

FIG. 4 illustrates an alternative x-ray imaging device in accordance with the present invention; and

FIG. 5 is an enlarged cut-away perspective view of a portion of a large area flat panel display in accordance with the present invention.

The x-ray imaging machine illustrated in FIG. 1 consists of an x-ray source 1 and a patient bed 2. As illustrated, the x-ray source 1 is movable so as to enable different regions of the patient bed to be selectively exposed to x-rays from the source 1. The patient bed 2 has incorporated within it a large area pixellated x-ray detector 3 which preferably extends substantially the entire length and breadth of the patient bed. Alternatively, the detector 3 may be smaller in area then the patient bed and preferably be mounted on a mobile support (not illustrated) located within the bed so that the position of the detector relative to a patient on the bed can be adjusted.

The detector 3 comprises an outer evacuated chamber 5 within which is mounted a monolithic array of electron multiplier channels 6 that is described in greater detail below. The surface 7 that supports the patients to be imaged, as illustrated, is or overlies the upper wall of the chamber 5. Within the upper wall of the chamber 5 or adjacent to the upper wall of the evacuated chamber is a scintillator 8 which generates photons in response to x-rays incident on the scintillator. A layer of photosensitive material 9 is then provided on either the lowermost surface of the scintillator 8 or on the upper surface of the array of electron multiplier channels 6 facing towards the scintillator 8. The electron multiplier channels 6 are open towards the scintillator 8 and have anodes 10 at the ends of the channels remote from the scintillator 8. Ideally, each channel has its own individually addressable anode 10 as illustrated. However, it is also possible for the x-ray detector 3 to include an array of individually addressable anodes 10 with each anode extending over two or more channels. In either case the array of anodes provides two dimensionally position specific data. The data from the anodes is fed via a power and data link 11 to an image processor 12 which converts the data into one or more images of the two dimensional x-ray intensity distributions detected by the detector 3. The anodes 10 and the electron multiplier channels are fabricated on a supporting substrate 13 all of which are encased within the evacuated chamber 5.

Where real-time x-ray imaging is required, it is possible to remove the substrate 13 and replace the anodes 10 and with a layer of phosphor so that the array of electron multiplier channels function as an amplifier of the original image generated by the scintillator 8.

The x-ray detector 3, which is illustrated in greater detail in FIGS. 2 and 3, consists of an array of electron multiplier channels 6 (only one channel is illustrated in FIG. 3) each of which has a respective individually addressable anode 10 located at the bottom of the channel. The top or head of each channel, facing towards the scintillator 8 is provided with photosensitive material 9. The photosensitive material 9 is generally selected on the basis of the wavelength(s) of light generated by the scintillator 8 and includes but is not limited to bialkali materials. The upper wall of the evacuated chamber 5, adjacent the scintillator 8, preferably includes a thin film of indium-tin-oxide (ITO) so that a potential can be applied to the upper wall of the chamber without unduly affecting the transmissive properties of the upper chamber wall to either x-rays or light in dependence upon the positioning of the scintillator 8 within or outside of the chamber.

Thus, the detector 3 includes internal gain as the electron multiplier channels 6 act to amplify the image generated by the scintillator 8. This enables much lower doses of x-rays to be employed in medical imaging without any loss of resolution and contrast in the final image generated by the detector. Moreover, as will be described in greater detail below, the fabrication methods for the detector enable large area pixellated detectors to be fabricated. For example detector surface areas upwards of 1 cm², 50 cm² and even over 1 m² are possible.

As the pixellated detector 3 of the present invention can be fabricated sufficiently large to enable the detector to extend the entire length of the patient bed as shown in FIG. 1. This means that with a movable x-ray source any area of a patient can be imaged with a minimum of movement by the patient. Indeed this enables the patient bed to act as a fixed reference point when taking repeated images or time sequenced images of a patient. Also, as the array of anodes is individually addressable this enables image data to be selectively collected from anodes. This can reduce the amount of image data being manipulated so as to increase the number of separate images that can be sequentially recorded and viewed in real-time within a fixed period of time. This can be of particular importance for in-theatre radiography. It also means that the detector is capable of delivering different levels of image resolution so that a particular smaller area of interest can be re-examined using a tight x-ray beam and a higher resolution image generated of that area.

Although the above has been described in relation to medical imaging it will, of course, be apparent that the present invention is equally applicable to non-medical x-ray imaging including non-destructive testing (NDT) where large area detectors are required for example checking aircraft for micro-fissures.

The array of electron multipliers is similar in construction to that described in EP-A-1004134 by the same author, the contents of which is incorporated herein by reference. The detector 3 consists of a monolithic structure of alternatively stacked layers of a dynode material 14 and an insulator 15 on the substrate 13 with an array of channels 6 having been etched through the dynode 14 and insulator 15 layers. The dynode material is electrically conductive and is preferably a metal. However, other electrically-conductive materials suitable for use as the dynode material include, but are not limited to, high density graphite, pyrolytic carbon, rutile, doped alumina, doped zirconia or crystalline molybdenum. The channels 6 are etched so that they are staggered with the each dynode layer 14 projecting partially into the channels. At least a region of surfaces of the dynode layer 14 exposed in each channel are also coated in a secondary-electron emissive material 16 such as oxidised beryllium copper, lithium fluoride, sodium fluoride, sodium chloride, potassium chloride, rubidium chloride, caesium chloride, sodium bromide, potassium iodide, caesium dioxide or caesiated antimony.

Each dynode layer 14 also includes power connections so that a voltage may be applied to the layer. The voltage level applied to an individual dynode layer is dependent upon the position of the dynode layer in the stack of dynode layers such that increasing potentials are applied to the dynode layers in a direction towards the anode 10. Furthermore, each of the individual anodes 10 in the electron multiplier array includes a signal connection 17 for carrying an amplified signal representative of the amount of light incident on the photosensitive material 9 at the head of each channel. Both the power connections and the signal connections 17 extend through the evacuated chamber 5 for connection to the power and data link 11 and thence to the image processor 12.

The channels 6 of the electron multiplier array are preferably arranged in a regular grid formation with a spacing of between 10 and 500 microns, preferably less than 100 microns. However, the arrangement of the channels and their associated anodes can be varied subject to the requirements of the detector.

The apertures in each dynode layer 14 preferably have a diameter of between 10 and 100 microns. However, apertures having diameters between 1 and 1000 microns are also envisaged. The size of the apertures in the insulator layers 15 are greater than those in the dynode layers 14 and are preferably between 20-110 microns, though again apertures of diameters between 5 and 1100 microns are envisaged. In having larger apertures in the insulator layers 15, upper 18 and lower 19 surface edge regions of each dynode layer 14 are exposed. These exposed upper 18 and lower 19 surface edge regions ensure that, when an electron, generated by the photosensitive material 9, impacts the dynode layer 14, charge leakage across the dynode layer 14 is reduced.

The dynode layer 14 may be of any thickness greater than 1 micron and is preferably between 10 and 50 microns. The insulator layer 15 may similarly be of any thickness greater than 1 micron and is preferably between 10 and 50 microns. Moreover, the thickness of the dynode 14 layer is preferably that of the insulator layer 15. Ultimately, however, the thickness of each layer is a matter of design preference and may depend upon the desired characteristics of the detector, as well as the choice of materials for the dynode layer 14 and insulator layer 15.

The thickness of the secondary-electron emission material 16 is preferably between 10 nm and 200 nm. Moreover, the emissive material 16 preferably has a secondary-electron emission coefficient of at least 5.

Two methods of fabricating the detector will now be described, both of which employ micro-engineering techniques. In the first method, an array of convex elements are provided on a substrate. The convex elements are preferably a thermally deformable plastics material that adopts a generally convex shape following exposure to heat. A thin metal film having a low second secondary-electron emission coefficient, such as a chrome:gold alloy, is then deposited over the surfaces of the convex elements and the exposed surface of the substrate. The thin metal film is then patterned leaving the surface of each convex element covered by the metal film to form an array of anodes. Each of the anodes also has a power supply connection in the form of a thin strip of the metal film. A first insulator layer is then applied over the surface of the substrate and the anodes through a mask. The first insulator layer is patterned by the mask so as to provide a plurality of apertures each in the form of a channel aligned with and exposing a respective anode. A filler material, such as a polyimide, is then deposited into the apertures so as to completely fill the apertures and extend over the exposed upper surface of the insulator layer. The filler material above the surface of insulator layer is subsequently removed so as to expose the surface of the insulator layer. A dynode layer is then deposited over the insulator layer and filler material through a mask. The dynode layer is patterned by the mask with a plurality of apertures, each aperture being associated with a respective anode. The apertures in the dynode layer are then filled with the same filler material. The filler material is removed back to the upper surface of the dynode layer. The fabrication steps described above are then repeated to create an alternating series of insulator and dynode layers. A photosensitive material is then applied to regions of the uppermost layer immediately adjacent each channel. Finally, electrical connections are made to each anode and each dynode layer and the entire structure is sealed inside a steel or glass package under vacuum.

Preferably, after the filler material is removed so as to expose the upper surface of the insulator layer or the dynode layer, a continuous seed layer in the form of a thin film is deposited over the exposed surface of the insulator or dynode layer and the filler material. Preferably, the seed layer is of the same material as the dynode layer. The seed layers ensure that each layer in turn is planarised across the surface of the multiplier array thereby minimising any variation in the thickness of each layer across the array.

In a second, alternative method, the detector is fabricated by stacking a plurality of dynode-insulator plates. Each dynode-insulator plate is manufactured by bonding a layer of dynode material to a layer of an electrical Insulator. A mask defining a plurality of apertures is applied to the bonded layers and a jet of hard powders ablates corresponding apertures through both the dynode and insulator layers. The aperture walls in the insulator layer are thereafter selectively etched such that the apertures in the insulator layer have a greater diameter than those in the dynode layer. A layer of material chemically resistant to the selective etchant is preferably applied to the surface of the insulator layer remote from the dynode layer so that the thickness of insulator layer is maintained. The aperture walls of the dynode layer are then coated with a secondary-electron emissive material to create a single dynode-insulator plate having a plurality of apertures. A plurality of such dynode-insulator plates are then stacked together such that apertures in adjacent plates align to form a plurality of continuous electron multiplier channels. An end of the stacked structure is then closed by a substrate having a plurality of anodes, such as those described above for the first method, such that each electron multiplier channel is closed and has a respective anode. A photosensitive material is then applied to regions of the uppermost surface of the stack immediately adjacent each channel. Finally, electrical connections are made to each anode and each dynode layer and the entire structure is sealed inside a steel or glass package under vacuum.

The fabrication methods described above produce large area detectors that are sufficiently robust to be suitable for construction within a patent bed or as mobile detectors in the case of NDT. Hence, unitary monolithic arrays of electron amplification channels can be made with a continuous, active surface area, i.e. that surface of the array facing towards and responsive to incident electrons, of 1 m² or more. Moreover, these fabrication methods enable large area detectors to be fabricated that are capable of flexure and so can be integrated into non-planar structures. An example of this is illustrated in FIG. 4 which shows a patient bed with raised sides with a single, unitary and continuous large area detector supported by the frame of the bed to describe a non-planar detecting surface so that three dimensional x-ray imaging of a patient can be achieved in conjunction with an x-ray source 1 adapted for motion for example about an arc in a plane substantially perpendicular to the length of the patient bed and substantially parallel to the width of the patient bed.

With these fabrication methods, the detector 3 is capable of image resolutions which are in excess of 10 times better than those currently achievable using amorphous silicon detectors. For example the detector 3 is capable of 10 pixels per mm, more preferably 50-60 pixels per mm, though higher and lower resolutions are also possible.

Although the detector 3 described above is specific to x-rays, it is possible for similar large area detectors in accordance with the present invention to be provided for the detection of other forms of electromagnetic radiation including visible light, UV light and bioluminescence. Applications for such large area detectors include machine vision in automated production and bioscience such as DNA testing. Where the large area detectors are being used to generate images from incident visible light including ultra violet light, the scintillator 8 may be omitted as appropriate selection of the photosensitive material 9 enables the material to be reactive to incident radiation at these wavelengths.

Currently, in many automated production lines banks of individual cameras are used to monitor the production lines. With the detector of the present invention a single large area detector extending over the entire width of the production line may be employed instead. This is of particular importance where a continuous planar material such as aluminium, steel or glass is being produced.

Large area detectors in accordance with the present invention may additionally be employed for environmental monitoring. The large area detectors can be mounted on vehicles including aircraft to monitor for signature wavelengths of electromagnetic radiation such as gamma radiation. For these applications position sensitive detection is not necessary and the individual anodes may be replaced by a single anode extending over the entire surface of the substrate of the electron multiplier array.

In the case of bioscience, the large area detector can be constructed such that one or a cluster of electron multiplier channels correspond to each individual test site on a microarray use, for example, in DNA analysis. With a photosensitive material 9 sensitive to the particular wavelengths of fluorescence, the fluorescent signature of an individual sample can be quickly detected and as the electron multiplier channels act as amplifiers, even very weak fluorescence can be detected as the detector is capable of dynamic ranges of 5×10⁵.

The flexibility of the large area detector also makes it suitable for use as a fixed 360° camera. The detector can be shaped so as to describe a cylinder with the open ends of the electron multiplier channels facing outwards. In a preferred embodiment, focusing of the individual pixels is achieves by means of a cylindrical glass rod that is positioned close to the curved outer surface of the detector and substantially parallel to the central axis of the cylinder. The glass rod is movably mounted on a track that encircles the cylindrical detector so that the glass rod can be selectively positioned immediately in front of any surface strip of the detector. With this arrangement selective images of the area surrounding the camera can be recorded by means of the detector through appropriate positioning of the glass rod in front of the relevant pixels. Also, continuous images in selected or all directions from the camera can be recorded by causing the glass rod to continuously move around the circumference of the detector cylinder and synchronising the timing of data collection from any particular strip of pixels to when the focusing glass rod is in position.

The technology described above is equally well suited to large displays and in particular as a substitute for the far more expensive plasma screen technology. An example of a large area flat panel display is illustrated in FIG. 5. The display 20 comprises an outer evacuated chamber 5′ which is provided with a layer of phosphor 21 at an inner surface of one of the chamber walls (the phosphor layer 21 is shown as transparent In FIG. 5 to aid in understanding only). Facing the layer of phosphor 21, on the side of the phosphor layer opposite to the chamber wall, is an array of electron multiplier channels 6′. The electron multiplier channels 6′ are open at both ends (the original substrate having been removed during fabrication) and beyond the ends of the channels 6′ facing away from the phosphor layer 21 is an array of molybdenum tips 22. Thus, the electron multiplier array 6′ is introduced between the molybdenum tips 22 and the phosphor layer 21 so as to amplify the electron beam generated by the molybdenum tips. The means for driving the tips 22 so as to generate images in the phosphor layer 21 are generally conventional.

It will of course be immediately apparent that to achieve full colour images, adjacent electron multiplier channels are preferably grouped into threes with each channel in a group aligned with a respective phosphor region selected from red/green/blue such that each group of three channels and their respective three phosphor colours corresponds to a single display pixel.

The structure of the displays makes them particularly suited to large area displays. With the present invention it is possible to construct extremely large area displays for use, for example, at sporting events by arranging a plurality of individual display tiles adjacent one another, with each individual tile being 1 m² or more in size, for example. The presence of the electron multiplier array provides additional strength to the display tile whilst still allowing the tile some flexure which makes it possible for such display tiles to be used as advertising hoardings on non-flat surfaces.

The display of the present invention is also suitable for implementation as a head mounted display. The display is much lighter in weight than other conventional forms of head mounted displays which is an important consideration when the displays are intended for extended use. In addition, the display can be adapted to enable a wearer of the display to look through the display thereby reducing the restriction on a wearers awareness of their immediate environment which is encountered with many conventional head mounted displays. In this regard, the display can be manufactured with a series of through channels in the electron multiplier array to permit the passage of light from beyond the display. It will of course be appreciated that the fabrication methods for the display, described above, are particularly suited to the addition of through channels in the electron multiplier array.

Thus, it can be seen that in accordance with the present invention large area detectors and displays become possible and practical using the fabrication techniques described herein. The applications for such large area monolithic detectors and displays are numerous as evidence by the examples described herein which are not to be assumed to be limiting. Rather the scope of the invention is as defined in the accompanying claims.

1. An electromagnetic radiation detector comprising a layer of a radiation sensitive material; an amplification device; and one or more signal collectors, the amplification device comprising a plurality of alternatively stacked layers of a dynode material and an electrical insulator, each dynode layer having exposed secondary electron emissive material and each stacked layer having a plurality of apertures which align with apertures in adjacent layers to form a plurality of electron multiplier channels extending through the stacked layers, and power supply connections to each dynode layer for applying a predetermined voltage potential to each dynode layer wherein the one or more signal collectors is positioned at the opposite end of the electron multiplier channels to the radiation sensitive material such that a signal from the radiation sensitive material of the detection of electromagnetic radiation is amplified in one or more of the electron amplifier channels before being collected by said one or more signal collectors.

2. A detector as claimed in claim 1, having a plurality of signal collectors with each signal collector being associated with one or more electron multiplier channels.

3. A detector as claimed in claim 1, wherein the walls of the apertures in each dynode layer are tapered towards the one or more signal collectors.

4. A detector as claimed in claim 1, wherein the one or more signal collectors are one or more anodes mounted on a substrate located at the ends of the electron multiplier channels.

5. A detector as claimed in claim 4, comprising a plurality of individually addressable anodes and wherein the detector further includes an image data link for communication with each of the anodes for receiving position specific image data from each of the anodes.

6. A detector as claimed in claim 1, wherein the one or more signal collectors contain phosphor and emit light in response to incident electrons from the electron multiplier channels.

7. A detector as claimed in claim 1, wherein the amplification device consists of a continuous monolithic array of electronic multiplier channels having an upper active surface extending at least 1 m².

8. A detector as claimed in claim 7, wherein the upper active surface of the amplification device is non-planar.

9. An x-ray imaging device comprising a layer of x-ray radiation sensitive material; an amplification device; and an image processor, the amplification device comprising a plurality of alternatively stacked layers of a dynode material and an electrical insulator, each dynode layer having exposed secondary electron emissive material and each stacked layer having a plurality of apertures which align with apertures in adjacent layers to form a plurality of electron multiplier channels extending through the stacked layers, power supply connections to each dynode layer for applying a predetermined voltage potential to each dynode layer and a plurality of anodes located at the ends of the electron multiplier channels, each anode being associated with one or more channels and having an image data link for supplying position sensitive image data to the image processor for generating a two dimensional image of x-ray radiation incident on said imaging device.

10. An x-ray imaging device as claimed in claim 9, having an image resolution of at least 50 pixels per mm.

11. An x-ray imaging device as claimed in claim 9, wherein the amplification device consists of a continuous monolithic array of electronic multiplier channels having an upper active surface extending at least 1 m².

12. An x-ray imaging device as claimed in claim 9, wherein the upper active surface of the amplification device is non-planar.

13. A patient bed incorporating an x-ray imaging device as claimed in claim 9.

14. A patient bed as claimed in claim 13, wherein the x-ray imaging device is in a fixed position in the bed and extends substantially the entire effective length of the patient bed.

15. A patient bed as claimed in claim 13, wherein the longitudinal sides of the bed extend upwardly from the bed surface and include a portion of the x-ray imaging device whereby three dimensional x-ray images of a patient on the bed can be generated.

16. A display comprising a layer of phosphor material; an amplification device; a plurality of field emission tips; and a driver device, the amplification device comprising a plurality of alternatively stacked layers of a dynode material and an electrical insulator, each dynode layer having exposed secondary electron emissive material and each stacked layer having a plurality of apertures which align with apertures in adjacent layers to form a plurality of electron multiplier channels extending through the stacked layers, power supply connections to each dynode layer for applying a predetermined voltage potential to each dynode layer wherein electrons emitted by the field emission tips under control of the driver device are multiplied by the amplification device before being incident on the layer of phosphor material so as to generate a two dimensional image in the layer of phosphor material.

17. A display as claimed in claim 16, having a minimum tile size of 1 m².

18. A display as claimed in claim 16, wherein the layer of strips of different colour phosphor material each phosphor strip being aligned with one or more electron multiplier channels.

19. A head mounted display including a display as claimed in claim 16.

20. A head mounted display as claimed in claim 19 further including through holes extending through the amplification device permitting the passage of light.
